(19) United States
(12) Patent Application Publication
(10) Pub. No.: US 2009/0254162 A1
(43) Pub. Date: Oct. 8, 2009

(54) Novel Medical Device Conductor Junctions

(75) Inventors: Thomas Quinci, Oxford, PA (US); John Squeri, Downingtown, PA (US); Gregory A. Boser, Richfield, MN (US)

Correspondence Address: GORE ENTERPRISE HOLDINGS, INC., 551 PAPER MILL ROAD, P. O. BOX 9206, NEWARK, DE, 19714-9206, US

(73) Assignee: Gore Enterprise Holdings, Inc., Newark, DE (US)

(21) Appl. No.: 12/324,018

(22) Filed: Nov. 26, 2008

Related U.S. Application Data
(63) Continuation of application No. 11/549,284, filed on Oct. 13, 2006.
Application No. 11/549,284 is a continuation-in-part of application No. 10/830,597, filed on Apr. 23, 2004.

(51) Int. Cl. A61N 1/04

(52) U.S. Cl. 607/115

(57) ABSTRACT

A method for making an elongate medical device includes coupling a conductive fitting to an elongate conductor and providing an opening through an insulative layer in proximity to the fitting in order to expose the fitting.

### CROSS-REFERENCE

This application is a continuation-in-part of U.S. patent application Ser. No. 10/830,597, filed Apr. 23, 2004, entitled NOVEL MEDICAL DEVICE CONDUCTOR JUNCTIONS.

### TECHNICAL FIELD

The present invention relates to elongated medical devices and more particularly to novel conductor junctions.

### BACKGROUND

Cardiac stimulation systems commonly include a pulse-generating device, such as a pacemaker or implantable cardioverter/defibrillator that is electrically connected to the heart by at least one electrical lead. An electrical lead delivers electrical pulses from the pulse generator to the heart, stimulating the myocardial tissue via electrodes included on the lead. Furthermore, cardiac signals may be sensed by lead electrodes and conducted, via the lead, back to the device, which also monitors the electrical activity of the heart.

Medical electrical leads are typically constructed to have the lowest possible profile without compromising functional integrity, reliability and durability. Often junctions formed between a conductor and other components included in leads, for example electrodes, can increase the lead's profile, therefore it is desirable to develop low profile junctions.

### BRIEF DESCRIPTION OF THE DRAWINGS

The following drawings are illustrative of particular embodiments of the invention and therefore do not limit its scope, but are presented to assist in providing a proper understanding of the invention. The drawings are not to scale (unless so stated) and are intended for use in conjunction with the explanations in the following detailed description. The present invention will hereinafter be described in conjunction with the appended drawings, wherein like numerals denote like elements, and:

FIG. 1 is a plan view of an exemplary medical electrical lead in which embodiments of the present invention may be incorporated;

FIGS. 2A-B are perspective views of portions of the exemplary lead according to embodiments of the present invention;

FIGS. 3A-B are plan views, each of a portion of a lead subassembly according to alternate embodiments of the present invention;

FIGS. 4A-C are schematics, each showing a step of an assembly method according to alternate embodiments of the present invention;

FIG. 4D is a section view of a lead assembly according to one embodiment of the present invention;

FIGS. 5A-B are section views showing steps of assembly methods according to alternate embodiments of the present invention;

FIG. 6 is a section view showing a step of an assembly method according to an alternate embodiment of the present invention;

FIG. 7A is a plan view of a portion of a lead according to one embodiment of the present invention;

FIG. 7B is a section view of a segment of the portion of the lead shown in FIG. 7A;

FIG. 7C is a plan view of a lead according to another embodiment of the present invention;

FIG. 7D is a section view of a lead according to yet another embodiment of the present invention;

FIG. 8A is a plan view of a lead subassembly according to one embodiment of the present invention;

FIG. 8B is a section view of a lead assembly according to another embodiment of the present invention; and

FIG. 9 is a perspective view of an alternate embodiment of a portion of a lead subassembly.

### DETAILED DESCRIPTION

The following detailed description is exemplary in nature and is not intended to limit the scope, applicability, or configuration of the invention in any way. Rather, the following description provides a practical illustration for implementing exemplary embodiments of the invention.

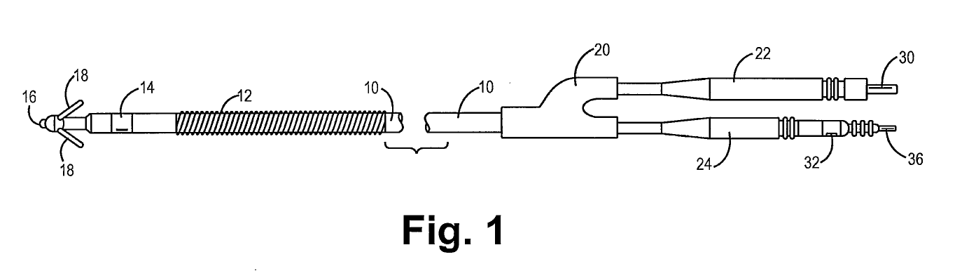

FIG. 1 is a plan view of an exemplary medical electrical lead 100 in which embodiments of the present invention may be incorporated. FIG. 1 illustrates lead 100 including a lead body 10 extending distally from a transition sleeve 20 to a distal end, which includes an electrode tip 16, tines 18 and an electrode ring 14; a defibrillation coil 12 extends along a portion of lead body 10 in proximity to the distal end. FIG. 1 further illustrates connector legs 22 and 24, which are adapted to couple lead to a medical device according to means well known to those skilled in the art, extending proximally from transition sleeve 20; conductors (not shown) extending through lead body 10, transition sleeve 20 and legs 24, 22 couple electrodes 16, 14 and 12 to connector contacts 36, 32 and 30, respectively, of connector legs 24 and 22. Embodiments of the present invention include means for coupling electrodes mounted about a lead body, for example defibrillation coil 12 or electrode ring 14, to a conductive wire or cable extending within the lead body.

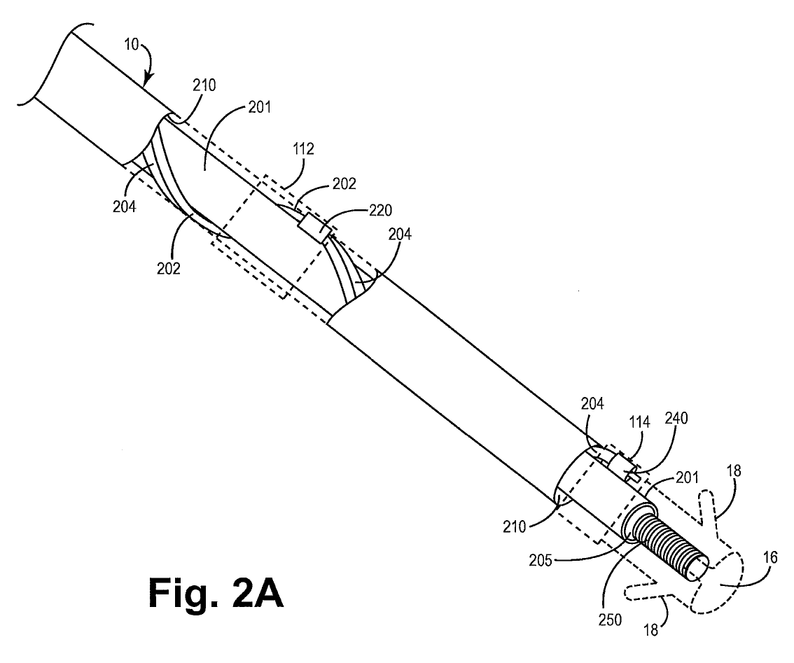

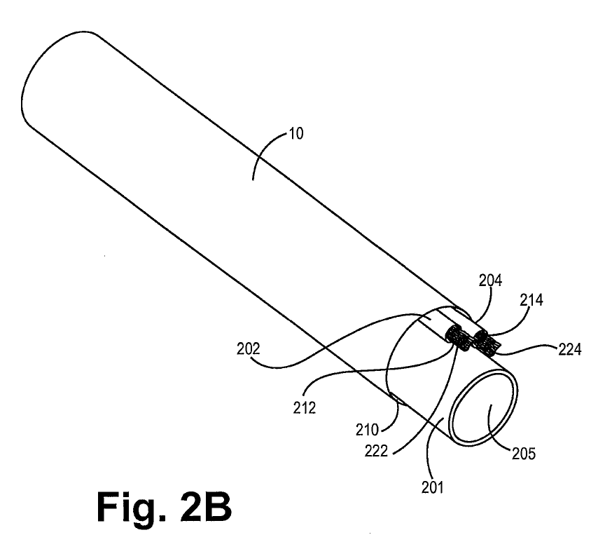

FIGS. 2A-B are perspective views of portions of the exemplary lead according to embodiments of the present invention. Via cut-away views, FIG. 2A illustrates lead body 10 including an inner elongate structure 201 about which a first conductor 202 and a second conductor 204 are positioned; a first conductive fitting 220 and a second conductive fitting 240 are coupled to first and second conductors 202 and 204, respectively. According to the illustrated embodiment, elongate structure 201 includes a lumen 205 in which an inner conductor 250 extends. According to an exemplary embodiment of the present invention lumen 205 has a diameter between approximately 0.01 and 0.03 inches. Using dashed lines, FIG. 2A further illustrates the extension of an outer insulative layer 210 over the subassembly, a first electrode 112 and a second electrode 114 coupled to conductors 202 and 204 via fittings 220 and 240, respectively, and a distal end of lead body 10 terminated by electrode tip 16, which is coupled to inner conductor 250, and tines 18. According to embodiments of the present invention at least first conductive fitting 220 is coupled to conductor 202, before covering the subassembly (formed, as illustrated, of inner elongate structure 201, conductors 202, 204 and fitting 202) with outer insulative layer 210. FIG. 2B illustrates a portion of lead body 10, according to one embodiment, before electrodes are coupled.

FIG. 2B further illustrates conductors 202, 204 each comprising a cable 222, 224, formed of a plurality of conductive wires bundled together, and an outer insulative layer 212, 214. According to alternate embodiments, conductors are each formed of a single wire; furthermore, although conductors 202 and 204 are shown wrapped or wound about inner elongate structure 201 in FIG. 2A, conductors 202 and 204 according to alternate embodiments can be positioned approximately linearly along inner elongate structure 201. An example of an appropriate material for conductor wires employed by embodiments of the present invention is an MP35N alloy; one or more conductor wires may further include a low resistance core, for example silver. An example of an appropriate material for insulative layers 212, 214 is ETFE, which may be formed as a jacket extruded about cables 222, 224 prior to positioning conductors 202, 204 along structure 201. According to some embodiments of the present invention, elongate structure 201 is formed from an insulative material, examples of which include fluoropolymers, silicones, and polyurethanes. It should be noted that when conductors 202, 204 are positioned along structure 201 they can be embedded in an outer surface of structure 201 according to some embodiments.

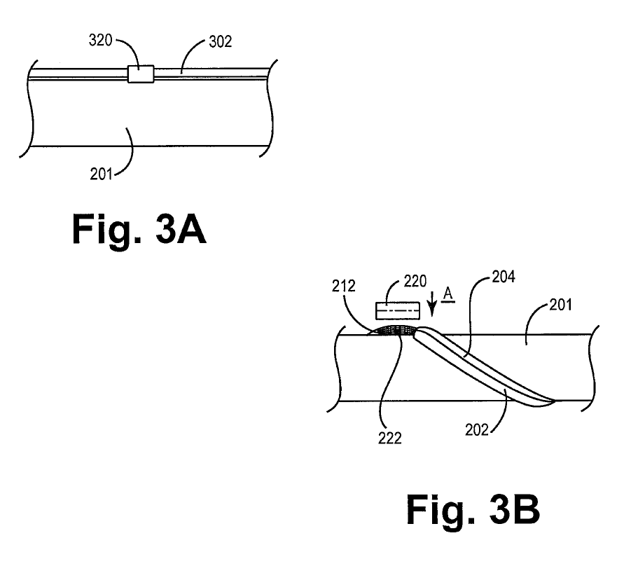

FIGS. 3A-B are plan views, each of a portion of a lead subassembly according to alternate embodiments of the present invention. FIG. 3A illustrates a subassembly of elongate structure 201 on which a conductor 302 including a conductive fitting 320 coupled thereto is positioned; according to this embodiment, conductive fitting 320 is coupled to conductor 302 prior to positioning conductor on elongate structure 201. FIG. 3B illustrates conductive fitting 220 being directed, per arrow A, toward a portion of conductor 202 where insulative layer 212 has been removed to expose cable 222 in order to couple fitting 220 to conductor 202; according to this alternate embodiment, fitting 220 is coupled to conductor 202 after conductors 202 and 204 have been positioned on structure 201. It should be noted that although FIG. 3B shows insulative layer 212 removed for coupling with fitting 220, other types of fittings having internal features to penetrate layer 212 may be employed so that layer 212 need not be removed for coupling. Furthermore, according to other embodiments of the present invention a fitting is coupled to a conductor, for example fitting 220 of conductor 202 (FIG. 2A), before an outer insulative layer, for example outer insulative layer 212 about cable 222 (FIG. 2B), is formed. According to these embodiments, the conductor and fitting are covered with an outer insulative layer, which is subsequently removed in proximity to the fitting, either before positioning the conductor including the fitting on elongate structure 201 or afterwards, and may be in conjunction with providing an opening in outer insulative layer 210. Means for removing the insulation in proximity to the fitting are well known to those skilled in the art and include but are not limited to, mechanical and laser stripping.

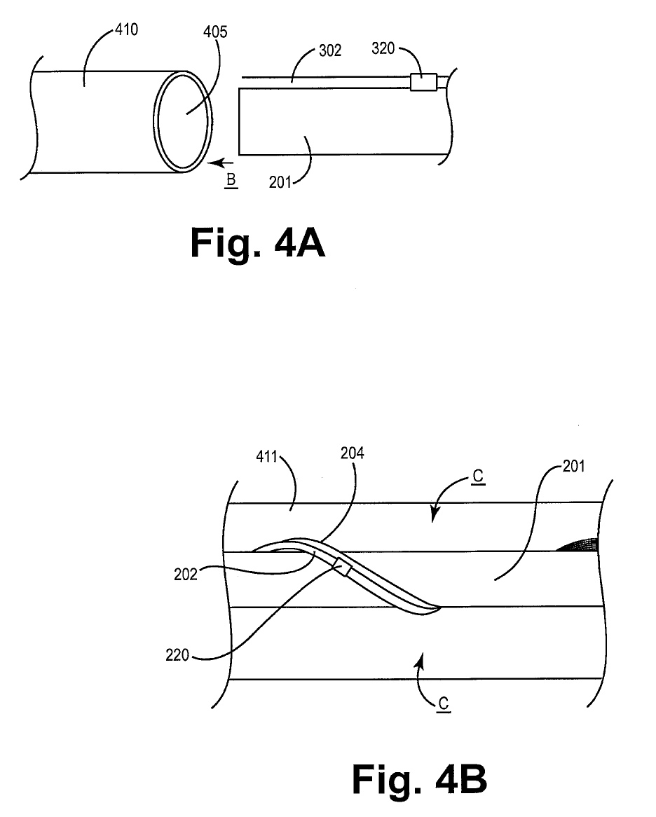

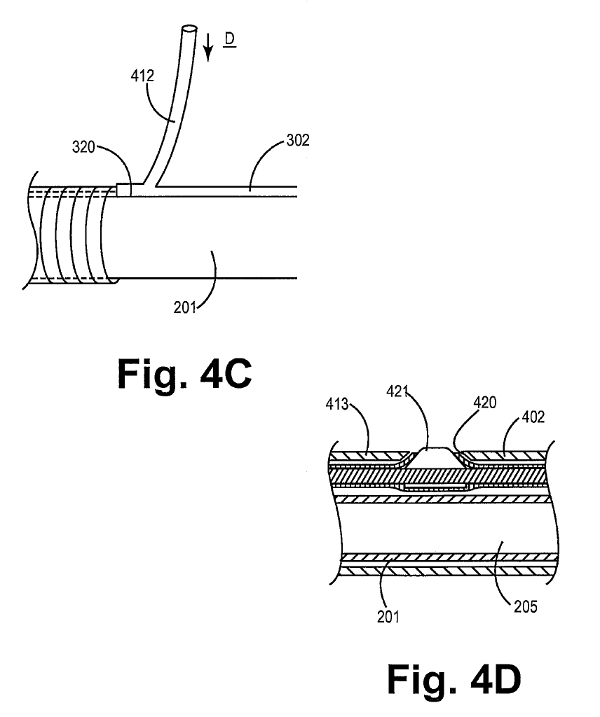

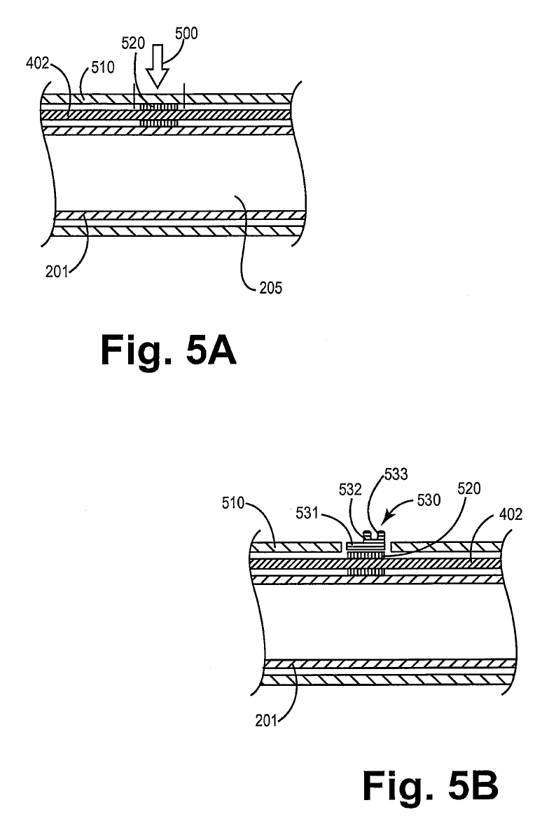

FIGS. 4A-C are schematics, each showing a step of an assembly method according to alternate embodiments of the present invention. FIG. 4A illustrates the subassembly shown in FIG. 3A directed, per arrow B, toward a lumen 405 of an outer insulative layer 410; according to this embodiment of the present invention, outer insulative layer 410 is formed as a generally tubular structure and the subassembly is inserted therein. FIG. 4B illustrates the subassembly shown in FIG. 3B, after fitting 220 is coupled to conductor 202, positioned in proximity to an outer insulative layer 411; according this other embodiment, outer insulative layer 411 is initially formed as a sheet and is wrapped about the subassembly per arrows C and then bonded along a seam formed when opposing edges of layer 411 come together. Suitable materials for layers 410, 411 include, but are not limited to, silicones, polyurethanes and fluoropolymers.

FIG. 4C illustrates the subassembly shown in FIG. 3A about which an outer insulative layer 412 is being wrapped per arrow D. According to yet another embodiment of the present invention, outer insulative layer 412 is in the form of a tape which is wrapped about the subassembly to form a lead body, the longitudinal edges of the tape being bonded or sintered together during or following the wrapping process. An example of a wrapping process is described in International Publication Number WO 02/089909 in conjunction with FIGS. 4 and 5; FIGS. 4 and 5 of WO 02/089909 along with associated descriptions therein are incorporated by reference herein. Although WO 02/089909 describes the process for covering a defibrillation electrode with e-PTFE, the inventors contemplate using the process in conjunction with an insulative fluoropolymer material to form outer insulative layer 412 according to some embodiments of the present invention.

FIG. 4D is a section view of a lead assembly according to an embodiment of the present invention. FIG. 4D illustrates a conductor 402 and a conductive fitting 421 coupled thereto positioned along elongate structure 201, and an insulative layer 413 including an opening through which a protrusion 421 of conductive fitting 420 extends. According to one method of the present invention, layer 413 is applied as a coating, either by an extrusion or a dip process, and the opening is formed during the coating process by means of protrusion 421 of fitting 420 penetrating through the applied coating. Referring back to FIG. 4C, an alternate method for forming an opening for fitting 320 is to leave an opening or a gap in the wrap of insulative layer 412. Suitable materials for layer 413 include, but are not limited to, silicones, polyurethanes and fluoropolymers.

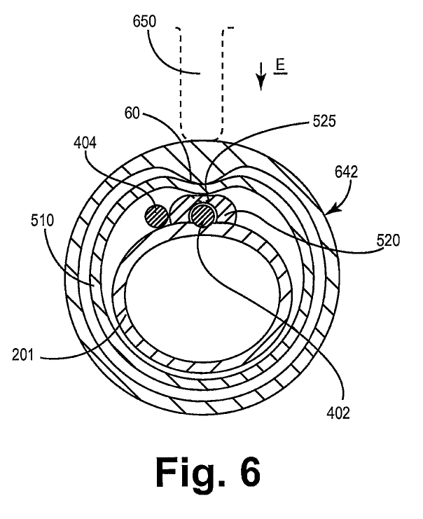

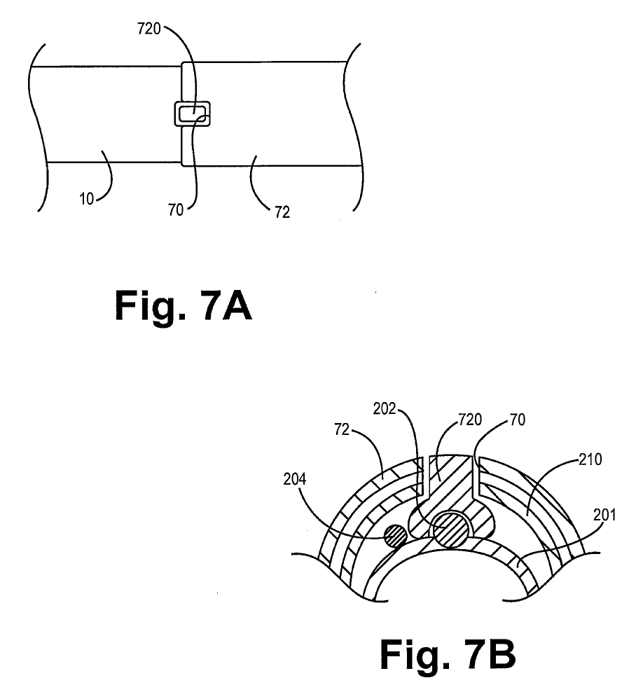

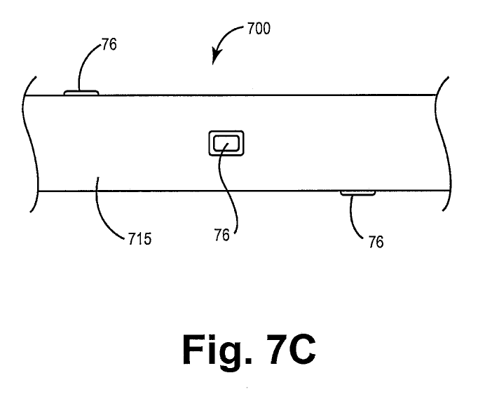

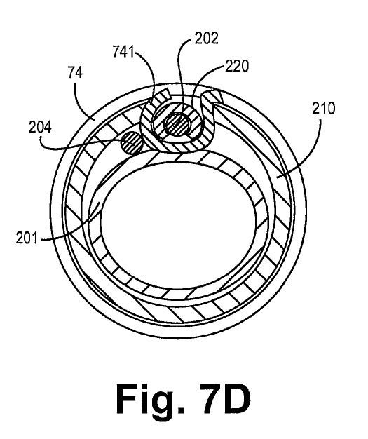

FIGS. 5A-B are section views showing steps of assembly methods according to alternate embodiments of the present invention. FIG. 5A illustrates a conductor 402 and a conductive fitting 520 coupled thereto positioned along elongate structure 201 and an insulative layer 413 formed thereover wherein a step to form an opening in proximity to fitting 520 is shown as arrow 500. According to one embodiment the opening is formed by mechanical cutting; according to another embodiment the opening is formed by ablation, i.e. laser; according to yet another embodiment an application of heat energy causing material flow forms the opening either independently or in conjunction with mechanical cutting. Means for forming the opening according to these embodiments are well known to those skilled in the art. FIG. 5B illustrates a subsequent step in an assembly method wherein, following the formation of the opening, fitting 520 is augmented with an attachment 530, which includes a protrusion 532 extending out through the opening to facilitate electrode coupling. According to the illustrated embodiment, attachment 530 further includes a portion 531 adapted for coupling with fitting 520, for example by welding, and a groove 533 adapted for coupling with an electrode, for example a filar of coil electrode 12 shown in FIG. 1. According to alternate embodiments, fitting 520 need not be augmented and an electrode includes an inwardly projecting feature to couple with fitting within or below opening; such embodiments are described in greater detail in conjunction with FIGS. 6 and 7D.

FIG. 6 is a section view showing a step of an assembly method according to an alternate embodiment of the present invention wherein forming an opening in proximity to a fitting is accomplished when an electrode is coupled to the fitting. FIG. 6 illustrates an electrode 642 mounted about a lead body formed by inner elongate structure 201, conductors 402, 404 positioned along the structure 201, conductive fitting 420 coupled to conductor 402 and insulative layer 510 formed thereover. FIG. 6 further illustrates electrode 642 including an internal feature 60 which is adapted to penetrate through layer 510 as a tooling head 650 is pressed against electrode 642 per arrow E; according to one embodiment, tooling head 650 is used for staking electrode 624 to fitting 520 and feature 60 penetrates by means of mechanical cutting; according to another embodiment tooling head 650 is used for resistance welding electrode 624 to fitting 520 by means of a current passed through head 650 and conductor 402 such that penetration is made via thermally assisted flow of material forming layer 510. Dashed lines in FIG. 6 illustrate a groove 525 which may be formed in fitting 520 and dimensioned to receive feature 60 of electrode as it is pressed inward; according to one embodiment groove 525 serves to facilitate the penetration of feature 60 through layer 510 which would be spread taught across groove during a previous assembly step.

FIG. 7A is a plan view of a portion of a lead according to one embodiment of the present invention and FIG. 7B is a section view of a segment of the portion of the lead shown in FIG. 7A. FIG. 7A illustrates electrode 72 mounted on lead body 10 and including a feature formed as a slot 70 into which a protruding portion of a fitting 720 is inserted for coupling, for example by laser welding. The section view of FIG. 7B further illustrates fitting 720 coupled to conductor 202 and the protruding portion of fitting 720 extending through an opening in outer insulative layer 210 to fit within slot 70 of electrode 72. FIG. 7C is a plan view of a lead according to another embodiment of the present invention wherein a protruding portion of fitting 720 includes an electrode surface 76 formed directly thereon, eliminating the need for an additional electrode component; as illustrated in FIG. 7C a plurality of fittings 720 may be positioned along a lead body 715 to provide multiple electrode surfaces 75.

FIG. 7D is a section view of a lead according to another embodiment of the present invention wherein a conductive fitting is inserted into an electrode feature for coupling. FIG. 7D illustrates an electrode 74 mounted on lead body 10 and including a hook-like feature 741 extending inward through the opening in outer insulative layer 210 to engage and couple with fitting 220, which is coupled to conductor 202. Hook-like feature 741 may be coupled to fitting 220 by means of crimping or laser welding.

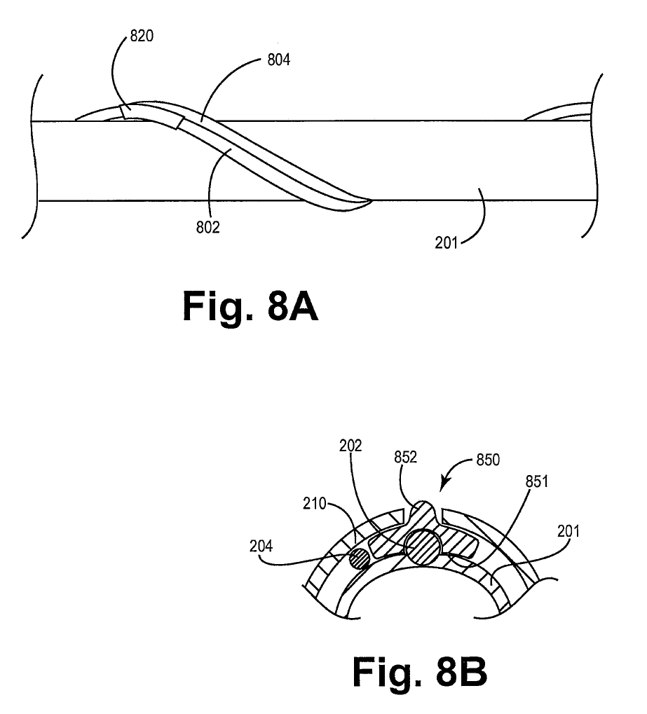

FIG. 8A is a plan view of a lead subassembly according to one embodiment of the present invention and FIG. 8B is a section view of a lead subassembly according to another embodiment of the present invention wherein fittings include surfaces conforming to a contour of the subassemblies. FIG. 8A illustrates the subassembly including inner elongate structure 201, a first conductor 802, a second conductor 804 and a flexible fitting 820 coupled to first conductor 802. Flexible fitting 820 may be formed of a conductive polymer, examples of which include intrinsically conductive polymers, such as polyacetylene and polypyrrole, and conductor-filled polymers, such as silicone rubber having embedded metallic, carbon, or graphite particles; once formed fitting 820 may be assembled about conductor 802 into a close fitting relationship, i.e. an interference fit, or fitting 820 may be formed in situ about conductor 802, for example by a molding process. Examples of metallic conductors, which may be used for any of the fitting embodiments described herein include, but are not limited to, platinum, platinum-iridium alloys, stainless steel and titanium.

FIG. 8B illustrates the subassembly including inner elongate structure 201, first conductor 202, second conductor 204 and a fitting 820 coupled to conductor 202; fitting 820 includes a surface 851 conforming to a contour of structure 201 and a protrusion 852 extending from an opposite side of surface 851 out through the opening in layer 210. According to the embodiments illustrated in FIGS. 8A-B positioning of conductors 802 and 202 about structure 201, after the fittings are coupled to the conductors, may be facilitated by the conforming fittings.

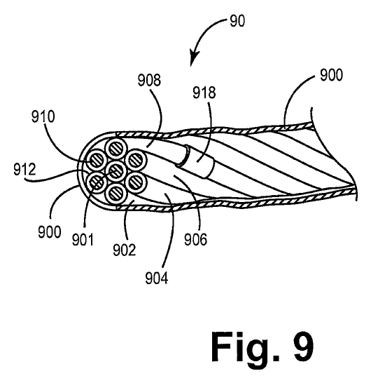

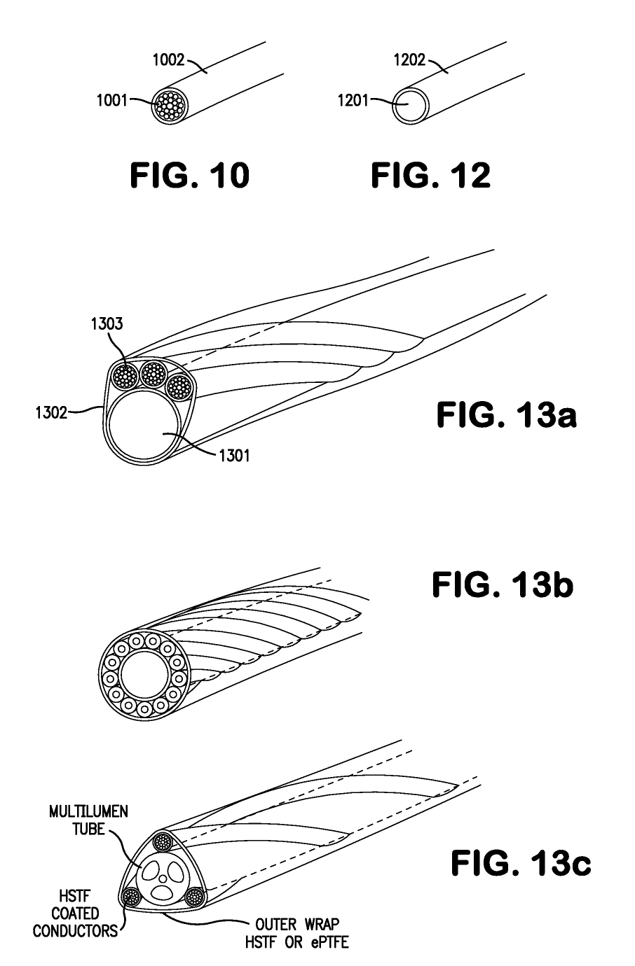

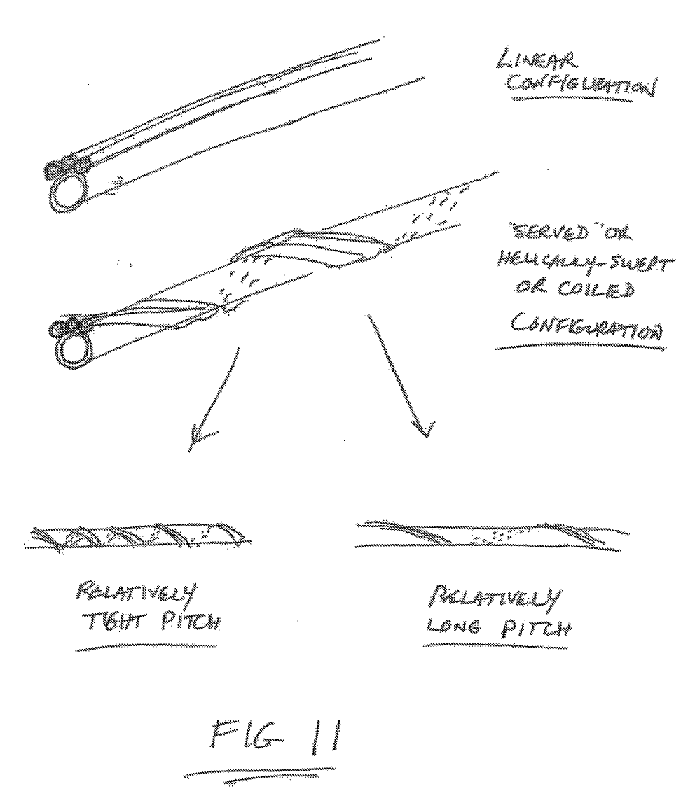

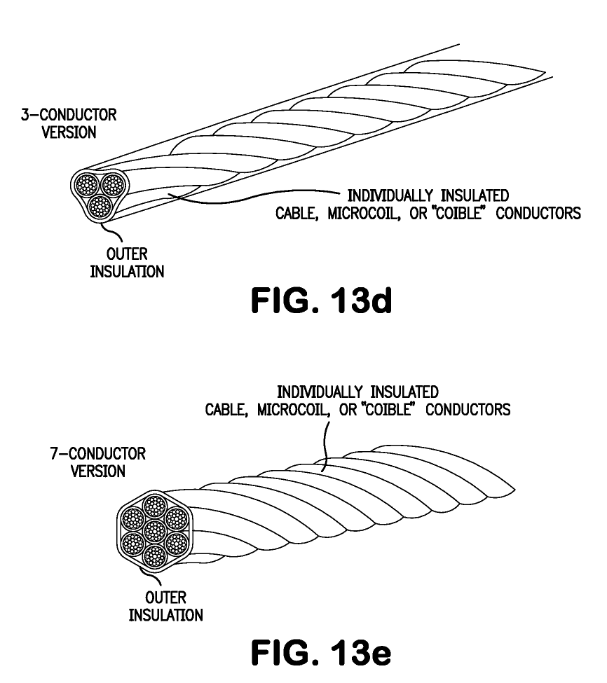

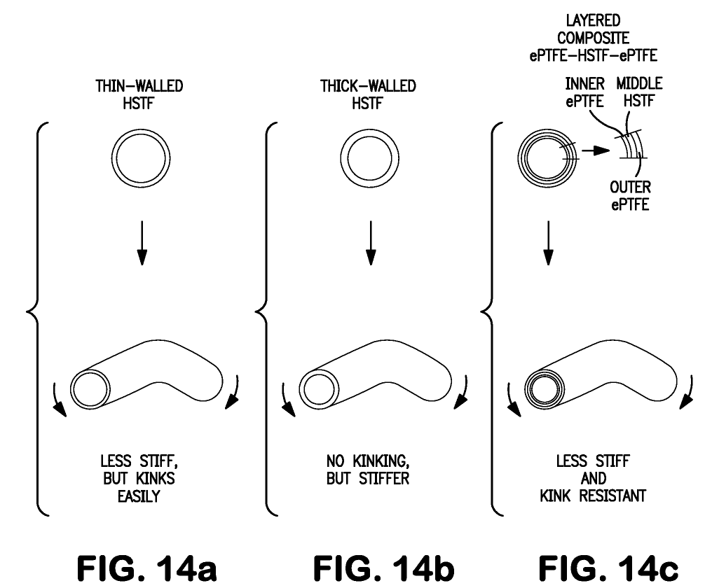

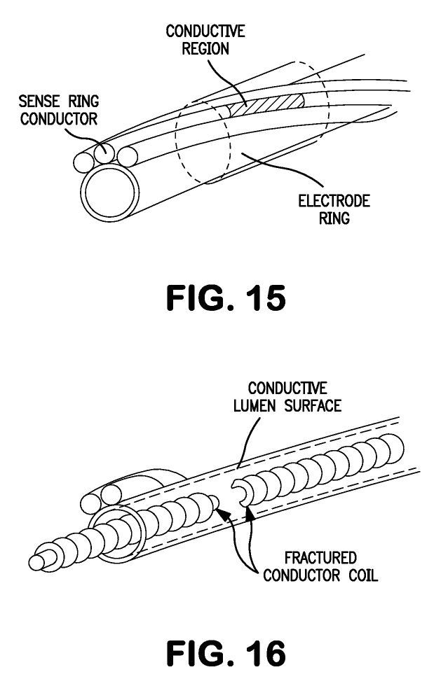

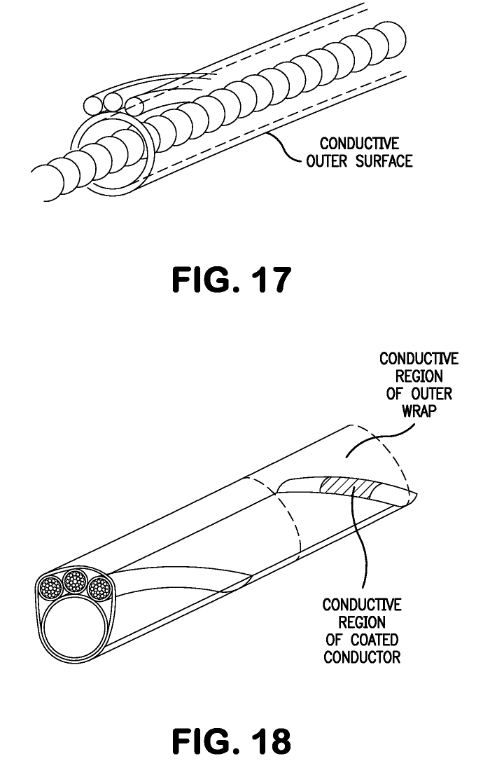

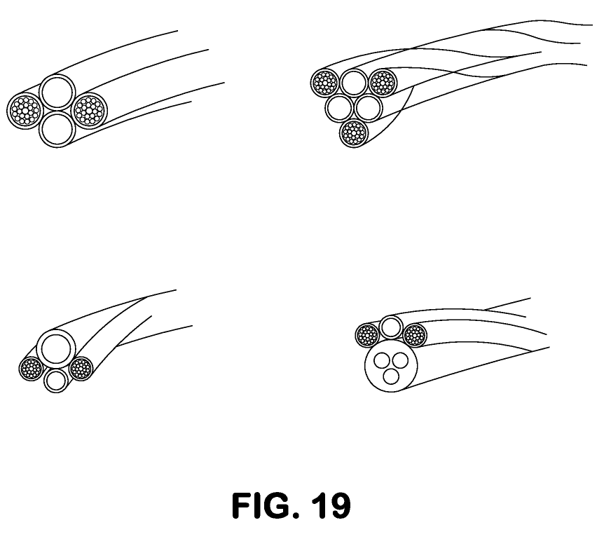

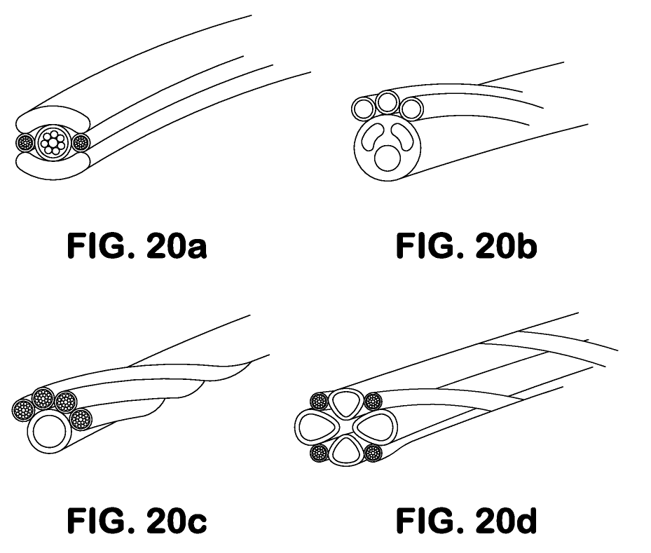

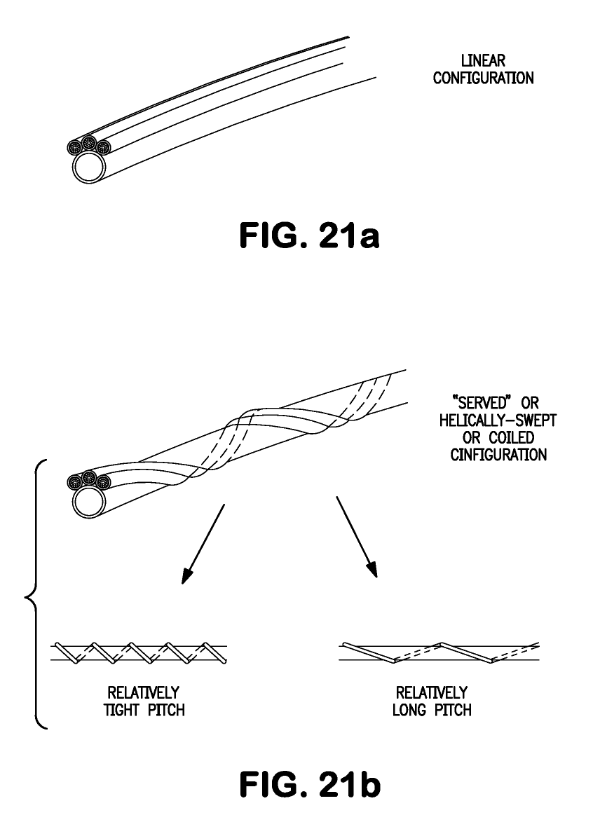

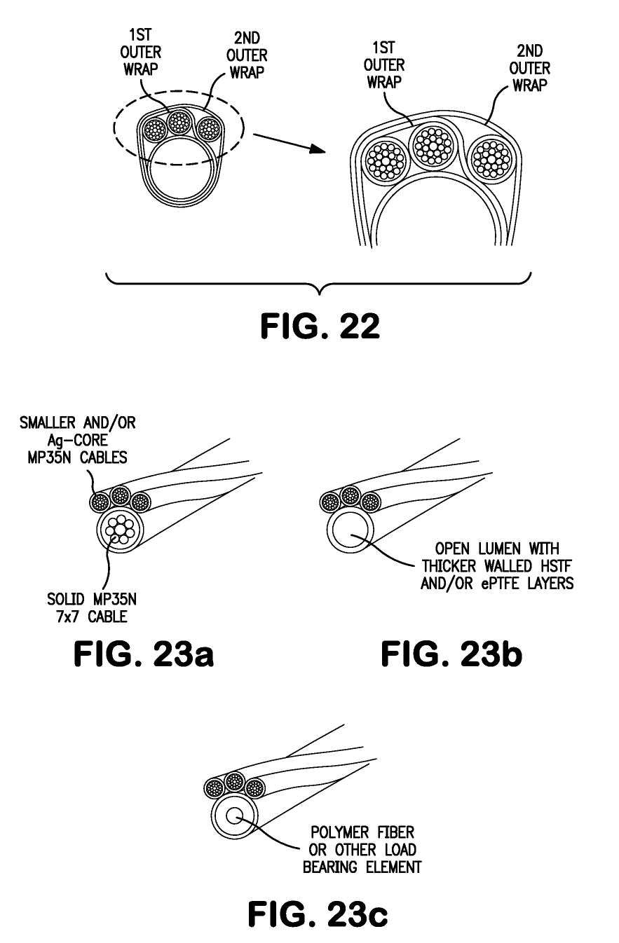

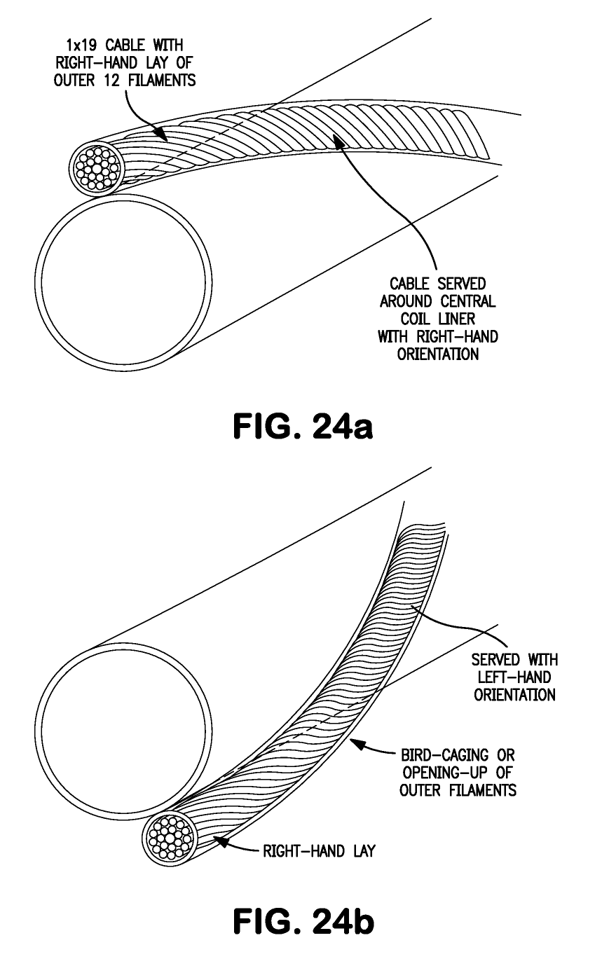

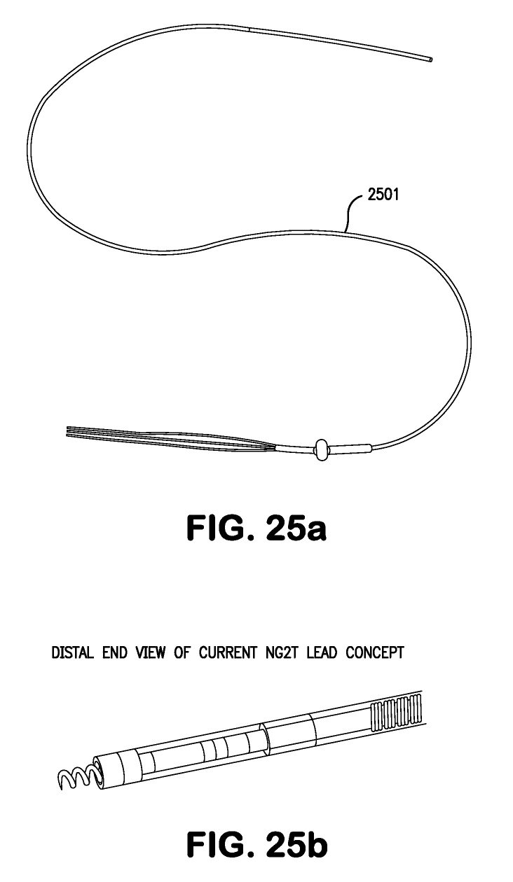

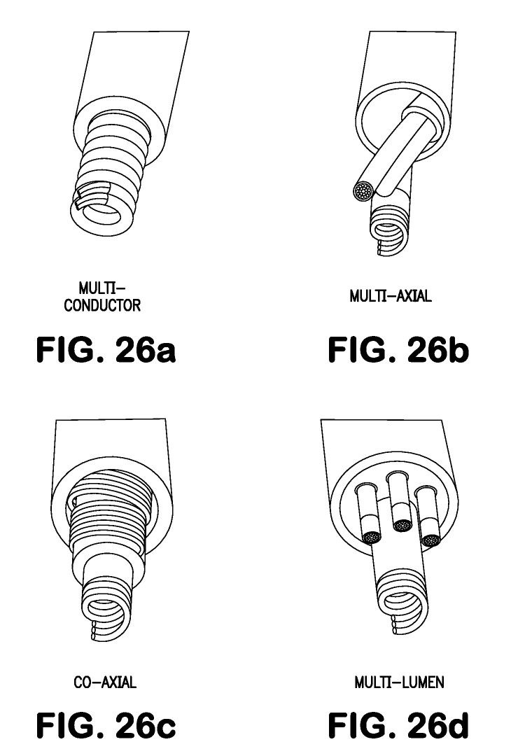

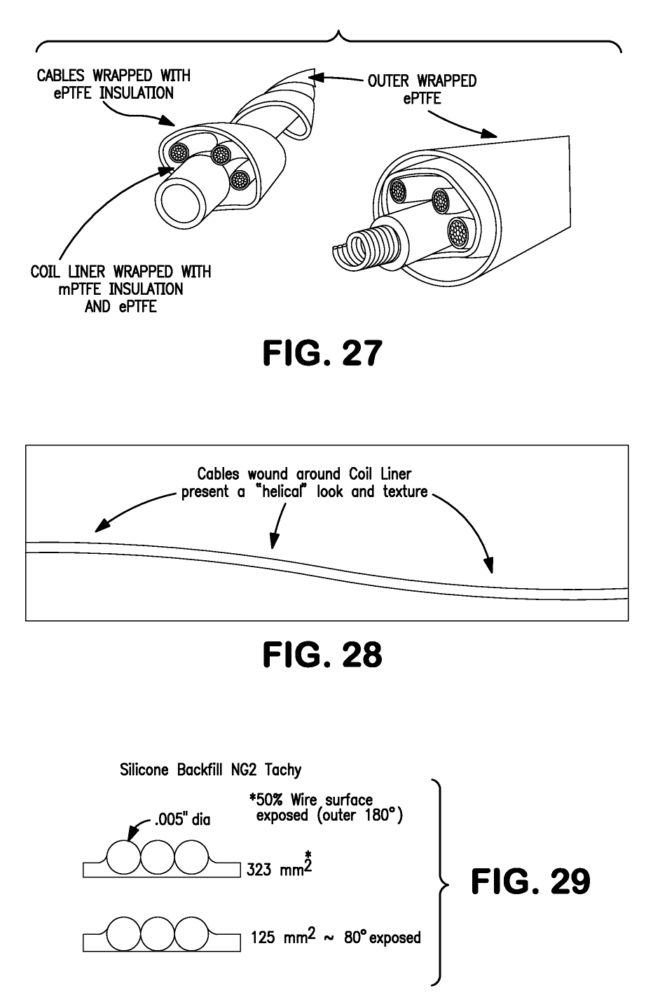

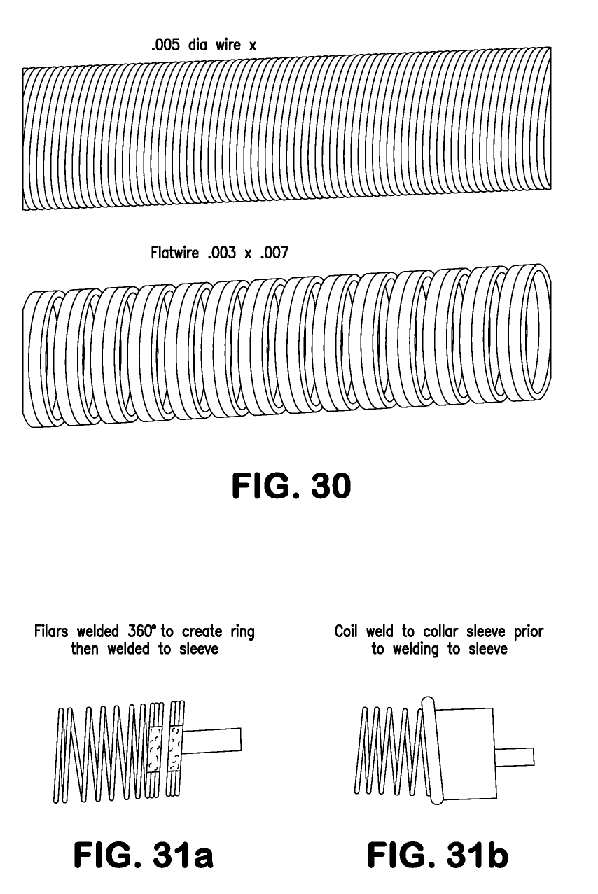

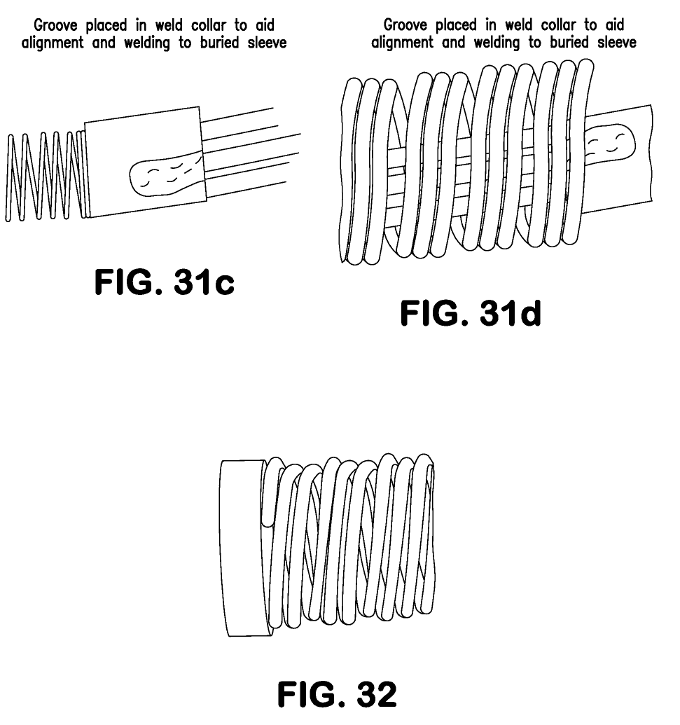

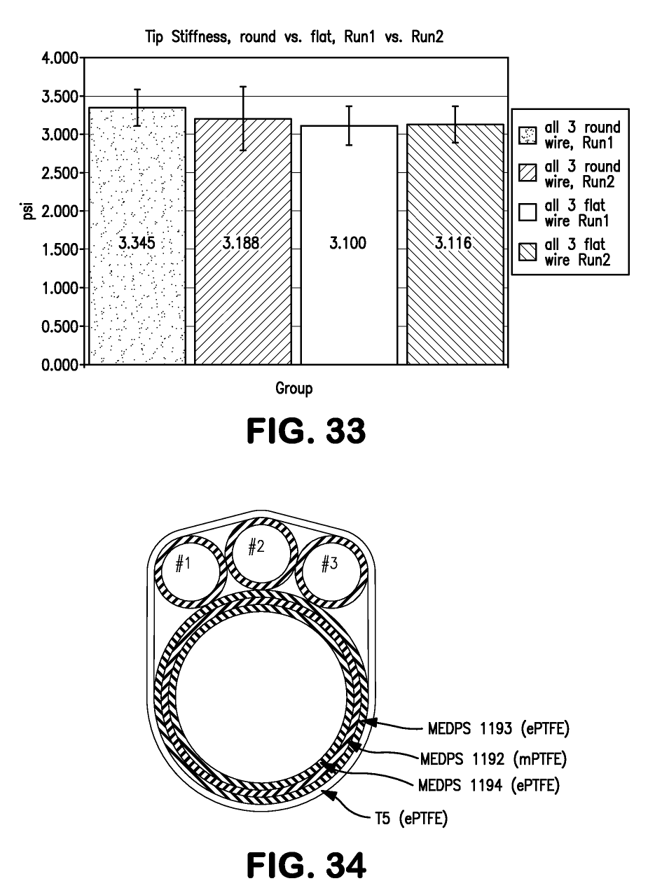

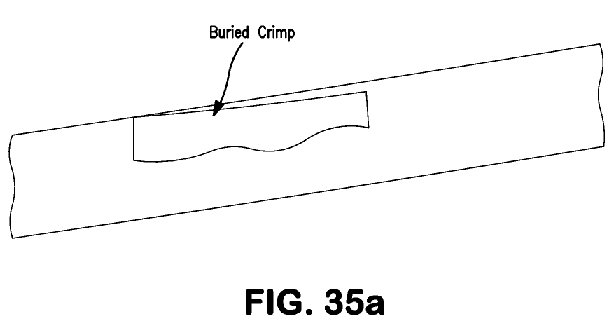

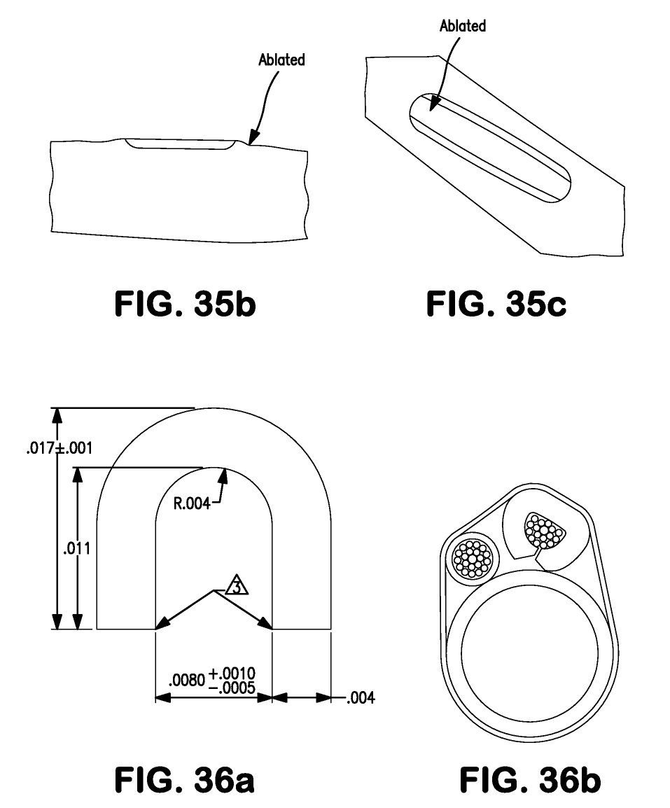

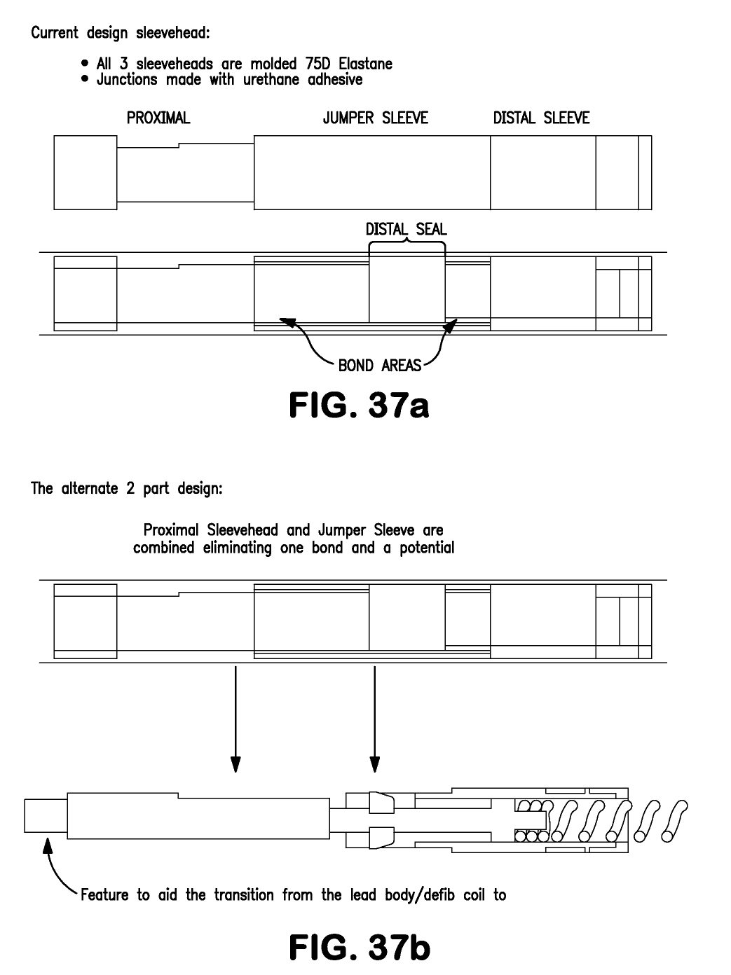

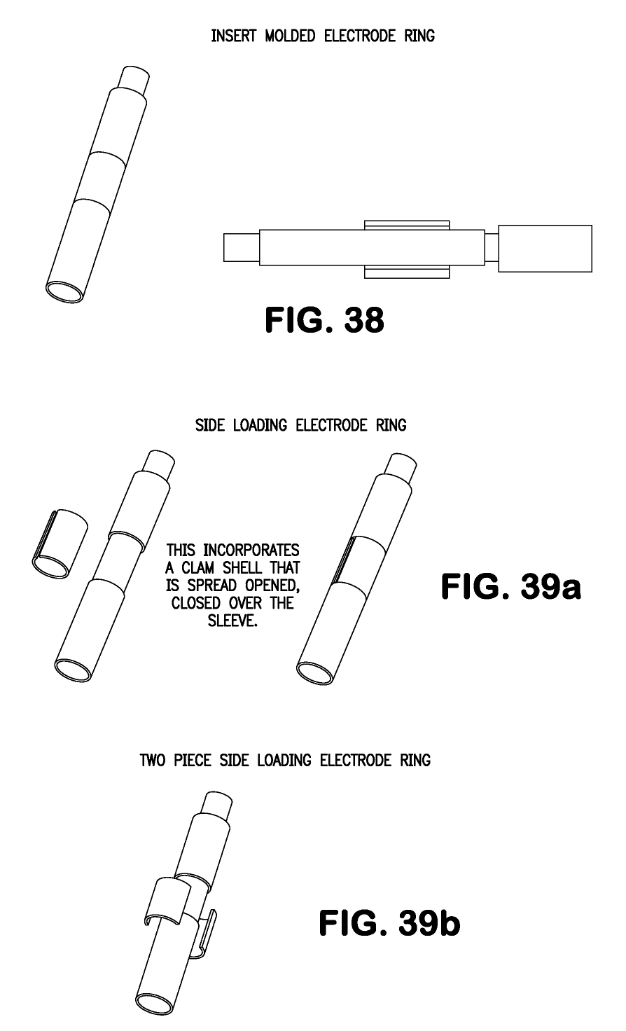

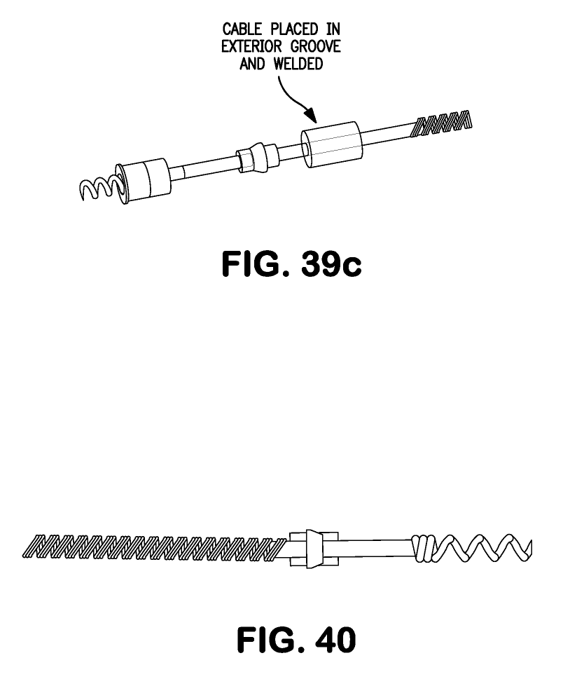

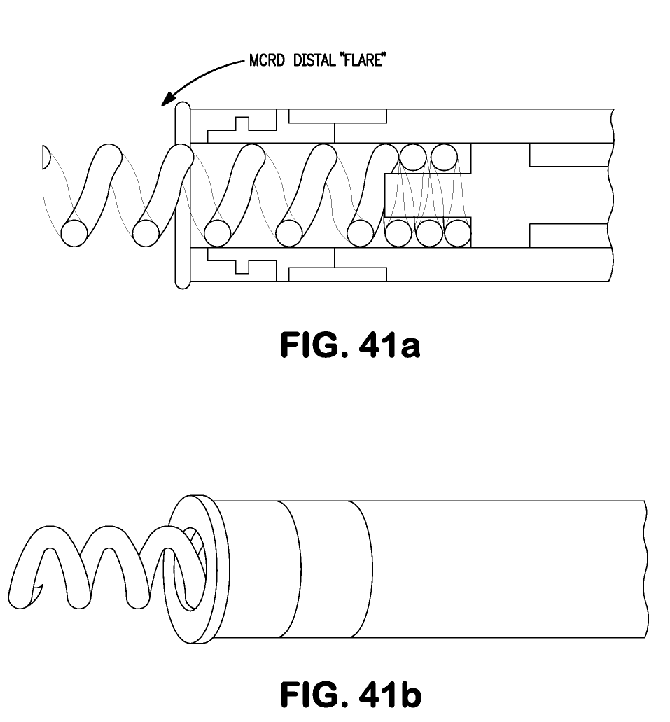

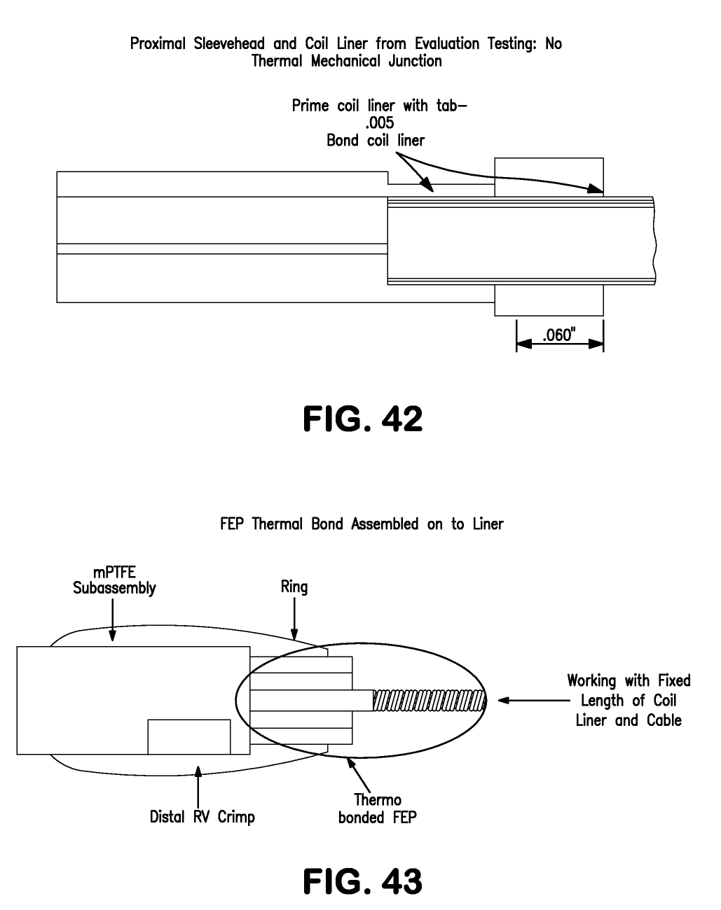

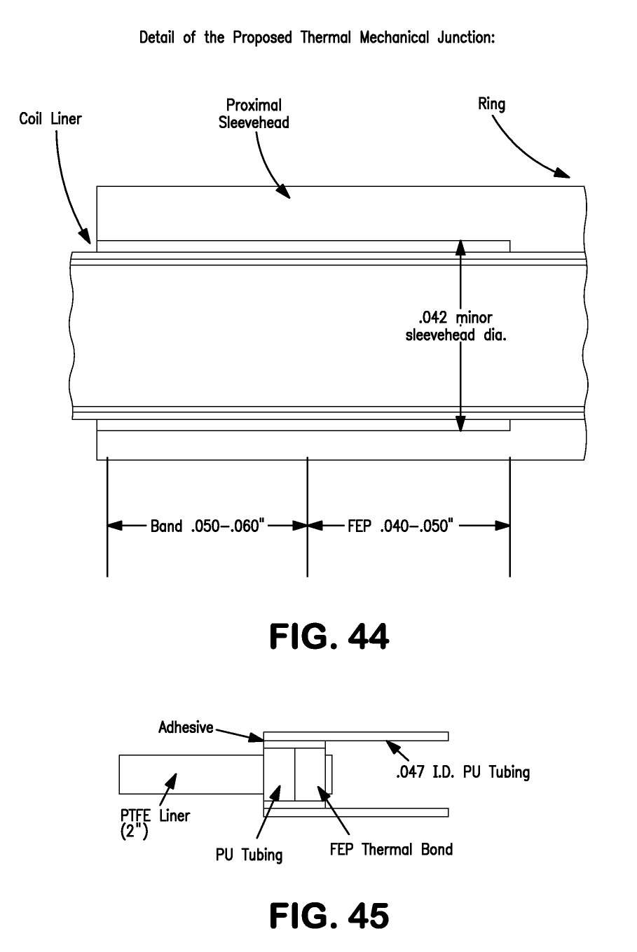

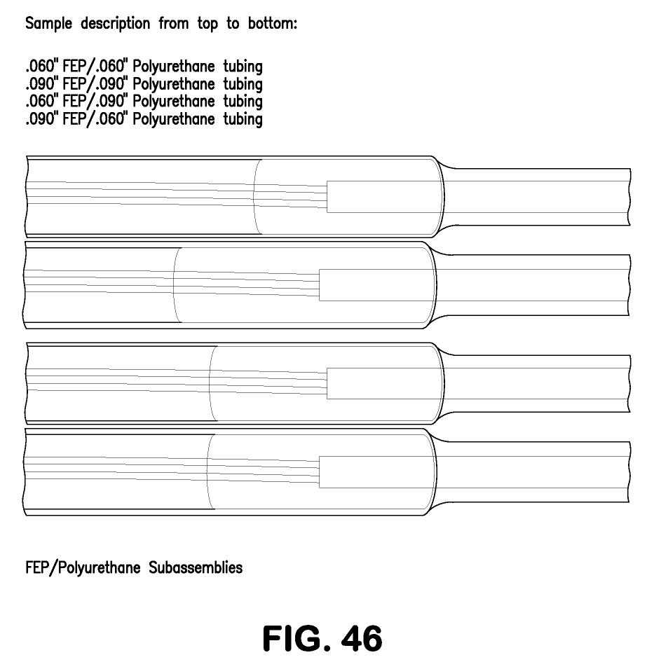

FIG. 9 is a perspective view of an alternate embodiment of a portion of a lead subassembly including a cut-away cross-section and a partial longitudinal cut-away section. FIG. 9 illustrates a lead body 90 including an insulative layer 900 covering an elongate structure 901 formed by an insulated conductor about which additional insulated conductors 902, 904, 906, 908, 910 and 912 are wrapped; a conductive fitting 918 has been coupled to conductor 908 prior to covering the subassembly with insulative layer 900. As previously described for other embodiments of the present invention, conductive fitting 918 may be coupled to conductor 908 either before or after positioning conductor along elongate structure 901; an opening subsequently formed in layer 900, either during or after the covering process, will expose fitting 918 for electrode coupling.

In each of the above described embodiments the openings through which couplings are made between electrodes and conductor fittings may be sealed with an adhesive, for example silicone medical adhesive or polyurethane adhesive, to prevent fluid ingress; sealing may be performed either before or after the coupling depending upon the embodiment.

It will be appreciated by those skilled in the art that while the invention has been described above in connection with particular embodiments and examples, the invention is not necessarily so limited; numerous other embodiments and uses are intended to be encompassed by the claims attached hereto. For example a host of other types of medical devices including electrical mapping catheters, ablation catheters and neurological stimulation devices may employ embodiments of the present invention.

Additional designs are disclosed for medical leads (e.g. next-generation (NG) VT/VF lead etc.) that employ fluoropolymer compounds. Fluoropolymer compounds are commercially available from W. L. Gore & Associates' Electronic Products Division in Elkton, Md. and Newark, Del. Other equivalent materials and processes produced by suppliers may be used.

The fluoropolymer materials include high strength toughened fluoropolymer (HSTF) and/or “expanded polytetrafluoroethylene (e-PTFE). In one embodiment, these materials are composed chemically of PTFE, but are mechanically modified to produce different physical morphologies, which in turn result in different mechanical and electrical properties. With respect to HSTF, the mechanical modification is done to provide enhanced mechanical properties such as tensile strength, abrasion resistance, and resistance to compressive creep or cold-flow, while maintaining a fully dense morphology and associated electrically insulative properties. Mechanical modification to produce e-PTFE on the other hand, results in a porous, open structure, which is not electrically insulative, but possesses comparable strength and abrasion resistance, and more flexibility and kink-resistance than HSTF. Both processes involve extruding and mechanically modifying the materials to produce thin (approximately 0.0002″) sheet, cutting the sheet into tape, and wrapping multiple layers of this tape around conductors, mandrels, and groups of previously wrapped conductors/mandrels to produce lead body subassemblies. In one embodiment, fluorinated ethylene propylene (FEP), a melt-processable PTFE copolymer, is used as a thermal adhesive to bond the layers together. Processing of HSTF and/or e-PTFE can be altered to produce differences in mechanical/electrical properties, including anisotropy in the mechanical properties.

Coated wire and cable components have been evaluated. Dielectric strength testing of HSTF in saline solution, after pre-soaking in IPA to more effectively wet any leak paths present, has shown coatings as thin as 0.0005″ to withstand up to 5000 volts of direct current (DC). HSTF coatings have been shown to have superior compressive creep resistance compared with extruded ETFE coatings.

Layers of e-PTFE can be bonded directly to HSTF to provide structural support, and has been shown to prevent kinking of a thin-walled open lumen tube such as a coil liner, without significantly increasing bending stiffness. Initial evaluations without e-PTFE indicated that although kinking of a coil liner could be reduced by increasing wall thickness to approximately 0.003″ this resulted in stiffness. A composite or layered coil liner, with HSTF on the inside and e-PTFE on the outside, resulted in lower stiffness, comparable size, and kink-resistance, while maintaining acceptable dielectric strength. Use of HSTF and/or e-PTFE Medtronic VT/VF platforms will enable significant downsizing the lead relative to platforms based on multilumen silicone and extruded ETFE insulations. Testing data has shown this material to have superior mechanical and electrical performance compared with extruded ETFE and PTFE.

1. The present invention significantly decreases lead body diameter, compared to lead bodies produced with conventional materials. For instance, with a lead body comprised of one coil with an ETFE liner, three 1×19 cables with extruded ETFE jacketing, housed in multilumen extruded silicone tubing, and a urethane overlay, the introducer size is currently limited to 7 French (Fr).
2. The present invention also performs better under compressive creep or cold-flow conditions, compared with conventionally produced PTFE and ETFE materials. For chronically implanted lead applications, appropriate insulation materials are needed that can withstand the mechanical loading conditions to the extent that electrical insulative properties are maintained for the duration of the implant. Fluoropolymer materials such as PTFE and/or ETFE produced via conventional means have been shown to have inferior creep or cold-flow properties compared with HSTF (e-PTFE may be better as well, although it's not used as an insulative layer). The superior mechanical/electrical performance of the HSTF allows lead body size to be reduced without compromising chronic reliability.
3. Fluoropolymer materials have excellent biocompatibility and chemical biostability properties.
4. The wrapped approach construction is better in terms of coating concentricity and processing-related loss of insulative properties (i.e. pinholes with thin extruded coatings), and is consistent with our business need to automate lead body assembly processes (i.e. eliminates stringing, lead body subassemblies cut-to-length or on-a-spool).
1. Exemplary medical electrical lead body configurations and attributes include, but are not limited to, the following as disclosed below:

### Lead Body Concepts

Examples of medical electrical lead body configurations and attributes include, but are not limited to, the following as disclosed below:
2. Basic configurations can include conductors 1001 (cables, microcoils, coibles, coiled cables etc.) which are individually wrapped with HSTF and/or ePTFE 1002 (FIG. 10), and open tubes or liners 1201 composed of HSTF and/or ePTFE 1202 (FIG. 12), e.g. to house coils, coibles, coiled cables, fibers, filaments, various types of torque wires or components capable of torque transfer, or to remain open to function as a compression lumens, deliver fluids, drugs, or biologic or other materials. Some typical configurations include, but are not limited to, that shown in FIGS. 13a through 13e.
a. The open tubes or liners 1301 are produced by wrapping HSTF and/or ePTFE tapes 1302 on a ductile mandrel, such as annealed silver-plated copper wire, and subsequently tensile pulling and uniformly necking down the mandrel for removal from the tube.
b. All these individual elements are then wrapped with HSTF and/or ePTFE tapes to produce a complete assembly, or alternatively, a subassembly which could be combined with other subassemblies to form a complete higher-level assembly.
c. All the individual elements and their outer wraps are thermally treated to sinter or bond the individual layers of HSTF and/or ePTFE together. This sintering or bonding can be accomplished by pre-coating or laminating the surfaces with FEP or other fluoropolymer adhesives, or by treating or modifying the surfaces with any other method which results in sintering or bonding between layers.
d. Bonding or sintering of surfaces other than that between layers can be done selectively, as needed. For instance, bonding between individual coated elements can be inhibited to allow relative movement, thereby reducing stiffness. Reduced stiffness can result in less trauma to the vasculature and cardiac tissue, and less risk of tissue perforation during implant and chronic use. An additional benefit of lowered stiffness is lower stresses in conductor and insulation materials.
e. Use of a thinner HSTF, or ePTFE instead of HSTF, for the outermost layer or “outer wrap” can result in reduced stiffness as well.
f. The degree of tightness with which the conductors/cables 1303 are “served” or helically swept or wrapped around a central coil liner tube can affect stiffness and degree of impingement on the coil liner. Impingement on the coil liner can affect the ease of stringing of coils, the ease of to insertion/withdrawal of a stylet, and the ability or effectiveness associated with torque transfer via rotation of a torque conductor coil. The stiffness of the cable materials and cable construct, the degree of residual stress in the individual filaments of the cable, and the residual torsional stress in the served cable, can also affect the degree of impingment on the coil liner. An understanding of the relative degree of impact associated with these factors is necessary to achieve a successful design and manufacturing process.
g. The tightness of the wrapped coating layers can be varied to affect easy of mechanical stripping, or ease of movement between elements, for instance to reduce bending stiffness and flex fatigue resistance.
h. The orientation of the wrapped HSTF and ePTFE layers can alternate between left and right-hand lay or serve, to produce more uniform torsional stiffness and “feel” of the lead body assembly.
i. Any of the wrapped coatings can also be composed of multiple types of materials, for instance alternating layers of HSTF and ePTFE, to affect mechanical or electrical properties. One embodiment can be a composite coil liner consisting of HSTF as the middle layer and ePTFE as the inner and outer layers. Although the ePTFE offers no insulation properties when wetted-out with a conductive fluid, it is more flexible than HSTF and when bonded to the underlying HSTF it can provide structural support or strain relief and help to minimize kinking of the HSTF when bent in small radii (FIGS. 14a-14c).
j. Any of the wrapped coatings or any of the individual layers of each of the coatings, can be made conductive either in selective areas, for instance to facilitate electrical conduction for connection to a component (electrode, connector ring etc.) (FIG. 15), or along the whole surface, for instance to produce a conductive lumen surface for redundant conduction when in contact with a conductor coil which has fractured (FIG. 16). Alternatively, the outer layer of a coated conductor or coil lumen can be made conductive, to facilitate shielding of electromagnetic interference (EMI) such as RF or MR energy (FIG. 17). Another configuration can be to make the coating conductive at selected regions along the coated conductor element, so as to serve as the electrical conduit to an electrode or to a conductive region in the outer wrap that functions as the electrode (FIG. 18). Coatings can be made conductive by compounding with an appropriate material such as carbon or metal particles, for instance Pt or Ta. Alternatively, coatings could be made conductive by depositing via plating, vacuum deposition, ion implantation, or other methods.
3. The individual conductor and tubular elements described above can be arranged in any number of ways, such as a central lumen to house a coil surrounded by coated cables or coibles (FIG. 13a). If a central lumen isn't required, a grouping of elements without lumens (i.e. cables or coibles) can be done (FIG. 13b). Alternatively, various configurations are possible if more than one open lumen is desired, for instance two or more smaller lumens, different sized lumens, or multilumen tubing (FIG. 19).
4. Any of the elements described above can also be of a non-circular cross-section, for example a kidney-shape or tear-drop-shape to better utilize the available space (FIGS. 20a-20d).
5. The elements on the periphery of the cross-sections can be longitudinally configured either linear or straight, or helically swept, “served”, or coiled around the central element with varying degrees of pitch (FIGS. 21a and 21b), or helically swept or twisted together if a central lumen isn't required (FIG. 13b). The pitch or degree of helical sweeping of the conductor elements can be increased to provide improved strain relief and fatigue resistance.
6. The outer wrap can be composed of several separate outer wrap sets, with each set effectively encapsulating each separate cable/conductor, thus providing redundant insulation (FIG. 22).
7. To facilitate electrical isolation of conductors, fluid sealing, and/or mechanical bonding, the HSTF and ePTFE surfaces can be treated via wet chemical techniques (i.e. Tetra Etch) or plasma techniques (i.e. Medtronic's plasma silane, atmospheric gas plasma, or equivalent processes). Treated surfaces can be done either selectively or on all surfaces, and can be done in tape form or after wrapping/sintering. With these techniques, standard silicone medical adhesive backfill methods can be used to bond and seal as required to provide electrical isolation, fluid leakage, and/or mechanical bonding.
8. Another method of facilitating electrical isolation, fluid sealing, and/or mechanical bonding for strength, can involve use of fluoropolymer or other adhesives. One example is the use of FEP or PFA in selective regions, which can provide effective bonding and sealing. With these materials, bonding could be accomplished during the normal post-wrapping bonding/sintering process (i.e. at the same time the HSTF insulation layers are bonded together), or as a post-processing approach during final lead body assembly. Examples include, but are not limited to, electrical isolation and fluid sealing around defibrillation connectors, and mechanical bonding and fluid sealing of the coil liner to the distal assembly. These approaches may allow minimization or elimination of backfilling with silicone medical adhesive.
9. The central element can be designed to sustain high tensile loads, for those applications that require it. For instance the central element can be a larger (i.e. 7×7) solid MP35N cable, surrounded by smaller Ag-core MP35N cables, coibles, or open lumens. Alternatively, the central element can be a thicker-walled HSTF or ePTFE tube (i.e. with tensile properties similar to “Glide” dental floss), or a tube to house a fiber such as ePTFE (ala “Glide” dental floss), polyester, LCP, UHMWPE etc. or extruded element such as PEEK, PEKK, or polysulfone or other suitable material, which is capable of sustaining the required loads (FIGS. 23a-23c).
10. The final lead body assembly can be housed in a silicone or polyurethane overlay tube. Besides using this approach to provide a protective jacket with other materials of proven biocompatibility and biostability, an overlay can be used make the lead body isodiametric, for instance to butt-up with the ends of the defibrillation electrodes.
11. Any of the conductors used in these configurations can have additional redundant insulations composed of chemically different materials. For instance polyimide coated wire, or anodized tantalum wire, can be used to produce coils/cables.
12. Color additives or use of different combinations of HSTF and ePTFE layers, to produce differences in appearance or contrast can be used to facilitate differentiation of circuits, either visually or via pattern recognition techniques.
13. In addition to using HSTF and ePTFE as tape materials (which are chemically composed of PTFE), ETFE or other suitable materials which can be produced in tape form and which has acceptable mechanical, electrical, biocompatibility, and biostability properties can be used. One advantage with using ETFE or other materials instead of HSTF/ePTFE, is to provide a structure which can be exposed to e-beam or any other irradiation process used for sterilization, without significantly degrading mechanical/electrical properties, i.e. PTFE is not as resistant to radiation as other materials.
14. Cables served with same orientation as outer filaments of cables are less prone to bird-caging (e.g. 1×19 cables with a right-hand lay of the outer 12 filaments should be served in a right-hand orientation around the central coil liner to prevent bird-caging or opening-up of the filaments) (FIGS. 24a and 24b).
15. Use of an ePTFE material for the inner layer of a coil liner, which is less “spongy” and less prone to shedding or “hairing” results in improved coil stringing, stylet passage, and helix extension requirements, e.g. material must be less prone to “piling up” or shedding of material with coil movement.
16.

### Lead Description

NG2 Tachy is a sub-5 French, extendible/retractable, stylet delivered, IS-4 connector lead body platform. The lead body 2501 (FIGS. 25a and 25b) uses modified polytetrafluoroethylene (mPTFE) and a new lead body design to reach a sub-5 French size. The lead body contains three cables running in a helical fashion from the proximal connector to the defibrillation coils and electrode ring. The cables are in a helical configuration for better flex life.

| | |
|---|---|
| Design | Quadipolar, Multi-Axial Lead |
| Handling/ | Traditional Stylet Delivery |
| Delivery | (.014) |
| Lead Body | Multi-axial, mPTFE Insulated |
| Description | Cables Wrapped Around a |
| | mPTFE/ePTFE Composite Coil |
| | Liner Bundled with a ePTFE |
| | Outerwrap, with Overlay Outer |
| | Tubing |
| Lead Body Size | 4.6-5 fr |
| RV coil length | 6.2 cm |
| Tip to RV coil | 12-13 mm |
| spacing | |
| Introd. Size | 5F |
| Cathode Surface | ~3.2 mm² |
| Area | |
| Anode Surface Area | ~10.3 mm² |
| Pace coil + | Coil- 6 filar MP35N (6949 Coil), |
| Insulation | Coil Liner- ePTFE/mPTFE |
| | composite, (Insulation 1 mil |
| | mPTFE) |
| Ring Cable + | 1 × 19 MP35N cable insulated |
| Insulation | with 1.5 mil of mPTFE. |
| Defib cables | 1 × 19 Ag cored MP35N with 1.5 |
| | mil of mPTFE. |
| Connector | IS-4/M-4 |

### Lead Body Subassembly Background/Concept Description

The NG2T Quadripolar lead is a lead that utilizes a modified fluoropolymer (mPTFE) for the primary insulation. The major benefits of using the mPTFE material include: thin layers of insulation which are mechanically robust, have high dielectric strength, and improved resistance to creep over traditional ETFE and PTFE. The use of these materials has also led to advances in manufacturing processing and a benefit to lead building. The mPTFE subassembly utilizes an outer ePTFE wrap to bundle the insulated cables and coil liner together. Windows and end cuts are made utilizing automated laser technology to prepare the subassembly for further manufacturing processing. A unique buried fitting approach (US Patent 2005/0240252 incorporated by reference in its entirety) provides the foundation for laser welding the defib coils to the subassembly. The method of assembly of the mPTFE insulation layers allows the fittings to first be crimped on the cables before insulation is layered over the cables and fittings. Upon completion of the subassembly, the fittings are then exposed with a small laser ablated window and minimize any unnecessary openings to expose the lead body. Furthermore, the skill, tools, time, and energy is no longer needed to string conductors through the multilumen, nor open the multilumen at multiple places to manipulate the conductors and cross-grooves.
The mPTFE material and subassembly provides the thin insulations necessary to produce a sub-five french lead, while still providing tough, creep resistant materials at very high dielectric strengths. An additional benefit of the mPTFE subassembly with the NG2T Quadripolar lead is the ability to utilize the Sprint Fidelis conductor coil for extension/retraction and the acceptance of a 0.014″ stylet.
The mPTFE subassembly is unique in its multi-axial design (FIG. 26b) compared to the current multilumen assembly used in transvene high voltage lead applications. The design allows a twisting, or serving, of the conductor cables around the coil liner producing a superior flexing lead body (reference FIGS. 27 and 28). Furthermore, the serve of the cables directly effects the subassembly, and therefore impacts the lead body, stiffness and drape for handling at implant. The inner conductor and cable conductors are all insulated with a modified poly tetraflouroethylene mPTFE).
The mPTFE has been mechanically modified to resist abrasion and creep and provide high dielectic strength at very thin layers. The mPTFE is assembled with a wrap process that provides tight tolerances of layers and pin-hole free insulative layers. The inner conductor coil liner is a composite of mPTFE and expanded PTFE (ePTFE) to provide electrical isolation as well as resistance to kinking and the lead handling characteristics. The cable conductors and coil liner are bundled together with an outer ePTFE layer. The outer, tissue contacting layer, is a protective non-insulative tubing used to aide in lead handling and provide isodiametric geometry for ease of venous entry and lead extraction. The overlay tubing may be made of SME polyurethane or PurSil co-polymer. The proximal connector will use an IS4 configuration to connect to a device. The lead accepts a 0.014″ (blue, grey) or smaller stylet.

### Defibrillation Coil Concept Description/Approach

A 7 french introducer and a 6.6 french lead body. Below is a table comparing MDT market released leads RV electrode designs for dimensions, surface and shadow areas to that of NG2 tachy.

| | CHART 1 | |
|---|---|---|
| | NG2 Tachy | |
| Diameter | ~4.6Fr | (1.5 mm) |
| RV coil length | 6.2 | cm |
| RV Surface Area | 323 | mm² |
| RV Shadow Area | 285 | mm² |

Silicone rubber backfill prevents in-growth of fibrotic tissue into and under the defibrillation electrode coil filars. Approximately 50%, 180° of the interior diameter, of wire surface to be covered with silicone adhesive. The remainder is wiped away during the manufacturing process leaving the outer surface, 180°, free of silicone rubber. See FIG. 29.
The quality of the embedment process can vary and may be difficult to evaluate visually. The larger wire size of previous ICD leads improves the manufacturability of the backfill process; larger surfaces are easier to clean. The smaller wire size of the NG2 Tachy creates smaller crevices that can retain silicone rubber. The figure above show the differences between a 180 backfill to an 80 exposed surface. The resultant area is reduced by over 60%.

It was concluded that the TXD lead design is capable of having adequate surface area for comparable defibrillation performance to previously release ICD leads.

In addition a flat wire approach (which eliminates the need to try to clean the crevices) and alternative embedment processes may be used.
A separate backfilled subassembly allows the defib coil to be embedded with a uniform substrate before stringing onto the lead body, which has a non-uniform diameter (cables wrapped around the coil liner are non-uniform) and also will allow the composite stiffness in the defibrillation coil region to be reduced (see Stiffness section).
Pre-backfilled coils examples are shown in FIG. 30.
Examples of 5 weld joint design concepts that have been explored for connecting the defib coil to buried crimp sleeve joint are shown in FIGS. 31a-31d (also see Buried Crimp Sleeve Section).
Welding the filars together to form a ring or attaching a ring to the defib coil (FIG. 32) are two most promising methods at this time. The Advanced Manufacturing Engineering group is also exploring a wire feed process that can add filler metal directly to the defib coils.

### Distal Lead Stiffness—Current Approach

Leads have been made that meet a 3.6 psi tip stiffness requirement. The lead body subassembly (LBS) was made with a cable pitch of 0.812″ and an ePTFE (T5) outer wrap material that was treated with FEP to adhere it to the cables and the coil liner. The SVC cable was then able to be peeled out of the LBS without losing the pitch or having to remove the outer wrap. The SVC cable was cut 0.5″ distal of where the SVC coil would be placed.
These leads had a defib coil that was backfilled as a separate subassembly using FEP tubing (0.049″ OD) as a mandrel. The FEP tubing was stretched and removed so that the defib coil assembly could then be strung onto the lead body. The subassembly was then bonded to the lead body only on the ends. Two different defibrillation coils were used, a 0.005″ round wire coil and a 0.003″×0.007″ flat wire coil. Both leads showed acceptable tip stiffness, per plan RATR1572. The summary chart is shown in FIG. 33.

### Lead Body Constriction—Current Approach

Constriction of the LBS can effect stylet passage and the number of turns to ext/ret the helix. Constriction of the coil liner is caused by the non-uniformity of wrapping the cables around the coil liner.
A 0.026″ tooling stylet is being used to assess constriction at the LBS level. 100% testing should be done during development. Current requirement is free passage (insertion and withdrawal) of tooling stylet. Implementation of low torsion modifications to the cable serving equipment and were successfully able to make stylets pass freely and also make them stick.

### Buried Crimp Sleeve—Current Approach

The lead body subassembly design incorporates a buried crimp sleeve used to make a weld connection from the defibrillation coil to the cable. To expose the sleeve for this connection a laser is used to ablate the over wrap and the mPTFE cable insulation layers. An example of the buried sleeve in the LBS assembly is shown in FIGS. 35a-35c.
The current crimp sleeve design is part number A08157-001. This crimp does not close well or uniformly, see FIGS. 36a and 36b. About 50% of the crimps received on the LBS are not able to be welded because the seam faces upward.
The approach is to re-dimension the crimp sleeve to allow for more uniform shape and reduced seam gap. Two different sizes of round titanium tubing have been ordered and will be evaluated with current tooling. The new sleeves will be 0.003″ thick and 0.050″ long because this is worse case from a welding and processing stand point.

### Distal Sleeveheads/Joint and Electrode Concept Description

The current concept has three sleeve head components. These are required for assembly purposes since the cables are part of the LBS and the ring electrode needs to be sandwiched. This results in multiple joints that need to be bonded and reduces the area in the sleevehead for coil liner bonding and places overlapping joints in areas that may be needed for MRI feature as project progress. An alternative two-piece design and an insert molded and/or two-part electrode is currently being designed for the next concept. This concept eliminates two joints that were previously located behind the seal and eliminates possibility of fluid leakage through bonded areas and incorporates steroid MCRD. Additional information on the prior sleeve design/assembly method and the proposed new design are shown in FIGS. 37a and 37b, respectively.
Potential advantages/features of the two part design concept;

Proximal Sleeve allows for the coil liner to extend past the electrode ring. Increased coil liner bond length and redundant insulation past the electrode ring.

Proximal sleeve has insert molded ring option and allows the cable to be directly welded to a groove on outside of the ring. This eliminates the crimping and weld operations utilized in current 3 part design.

Proximal sleevehead design incorporates a feature to aids postioning the defib coil and the transition from the lead body/defib coil to the sleevehead.

Integrated design eliminates joints in sleevehead

This two part design requires that the electrode ring be either insert molded into the proximal sleevehead (FIG. 38) or have features that allow it to be side loaded onto the proximal sleevehead and welded closed (FIGS. 39a-39c). This may be accomplished by either a two part electrode ring that is welded together at two points or an electrode ring with a hinge or slot that is welded at one point.

### Insert Molded Ring Design Advantages:

Insert molding reduces handling of the TiN coating on the electrode ring and does not require an additional welding operation to close a hinge as in FIGS. 39a-39c.

Space used for clearances between the ring and the sleevehead are not needed and can be incorporated into the wall thickness of the proximal sleevehead and the ring.

Minimizes damage to ring caused by additional welding and fixturing operations which are required for hinged and two part concepts.

Eliminates alignment and position requirements during lead assembly.

### Helix Design Options and Design Approach

The NG2T helix is smaller than the current HV leads.
The helix is planned to be supplied as a welded subassembly. This lead incorporates two novel C-Stops (FIG. 40) which are snapped onto the drive shaft prior to assembly.

### Steroid Concept Description/Approach

The distal sleeve will incorporate an MCRD that is bonded to the outer diameter of the sleevehead. The MCRD is based on the 4196 Lead MCRD (molded component with same silicone, steroid, and ratio).
Two MCRD variations are being investigated at this time (straight cylinder and a flare). One incorporates a flare at the distal end FIGS. 41a and 41b This increases the overall tip diameter to 0.065-0.068 and thus decreases the lead tip stiffness (psi). It has been designed to collapse in the introducer.
The design/placement of the MCRD directly at the tip should provide several advantages:
1) Continues the practice of placing the MCRD/Steriod directly at the implant site.
2) Provide a thicker “soft” tip to minimize injury
3) Allow for tip to be enlarged but still be introducer compatible.
4) It is also been observed that this soft MCRD design will flare open and become larger when pressed against an object. This may help to reduce the potential for tip penetration.
5) Wrap around design allows increased steroid volume (˜3×4196) and still allow the indicator ring to be positioned close to the tip.

### Proximal End/Connector Concept Description/Approach

The concept is to use existing IS-4 connector module (P/N M924431A-002) and design and process for Model 6949M as much as possible.

### Key Similarities to 6949M Design/Process

Use of IS-4 connector module from MECC, P/N M924431A-002

Use of 1×19 cables and crimp blocks (all design and process work related to the joints between the cables and the connector module apply)

Use of 6949M conductor coil (all design and process work related to the joint between the coil and pin applies)

### NG2 Tachy IS-4 Concept Difference

To compensate for the smaller lead body and to add additional strain relief, the NG2T adds a silicone

tubing bonded to
the overlay to make the transition to the IS-4 distal connector sleeve.

### Thermal Mechanical Joints and Adhesion to the LBS Concept

### Descriptions/Approaches

Evaluation testing was done for a Technology Phase review presented in January 2006. This design configuration (FIG. 42) did not incorporate the thermal mechanical junction. The proximal sleevehead was bonded to the MPTFE lead body using urethane primer and adhesive after plasma treatment. Composite torsion, tensile integrity, and tensile testing did not meet requirements.

### Proposed Thermal Mechanical Joint Concept:

### Concept Description:

A new thermal mechanical junction approach has been proposed. In the current process, a band or ring is strung onto the coil liner followed by a length of FEP tubing. Silicone tubing is dilated with heptane and slid over the top of the FEP and the band. The assembly is placed in the cavity of a thermal forming die and exposed to temperature for a set time duration. The silicone tubing is removed and the coil liner and FEP are cut to length. Alternate processing schemes in which the FEP is processed first (at higher temperature) and then a band or ring made of urethane or some alternative with a slot or hinge is assembled onto the coil liner and thermally processed at lower temperature are also options. FIG. 43 shows the current assembly process. A technical peer review of this concept was held on Aug. 31, 2006 (reference BL0015721).

### Assumptions (See FIG. 44)

This joint will be loaded in tension and will need to meet a tensile design target

This joint also will be tested in torsion

Plasma treatment of the coil liner/cable(s) will be necessary

Use of thermal/mechanical approach with a ring (metallic or other) and FEP tubing is needed to pass testing

Bond length and diameter necessary for strength can be designed into sleevehead to allow the distal end to fit through a 5 Fr introducer

Tooling capability to control and minimize FEP diameter to fit into sleevehead

Fixturing is needed to provide thermal isolation of cable and coil liner

Tip to ring spacing and tip to RV spacing (13 mm) is adequate.

### Fluoropolymer Mechanical Junction for Medical Electrical Lead

A piece part component made from FEP or PFA can be thermo-bonded onto another fluoropolymer such as PTFE or ETFE to create a useful junction on implantable medical leads. This thermo-mechanical joining process results in a strong adhesive-like bond between the polymers. The junction formed can be used as a tensile or torsional bearing member or as a feature for assembly to other components. Due to the difficulty of obtaining good adhesion to fluoropolymers such as PTFE, this process allows leads to achieve strong mechanical joints without adhesives. Welding methods like ultrasonic welding or laser may also allow joining of these flouropolymers types in place of thermo processing with traditional heating methods such as thermal die bonding or hot air fixtures.

The use of FEP as a thermal bonded component on our PTFE insulation achieves a very strong bond not obtainable with other types of adhesive bonds. The thermal bonded FEP component allows us to locate other lead components adjacent to the FEP and results in a joint that can have high composite tensile strength or potentially be used to transfer torque loads. The challenges posed by the chemical resistant and bond resistant nature of PTFE can be overcome with this FEP thermal bond technique.

Multiple distal joint designs using an FEP thermal bonded component on our PTFE liner have been developed that will allow our NG2 Tachy lead to have a strong distal end connection. High distal composite joint strengths will allow chronic lead extraction from patients with less risk of lead separation/breakage and facilitate easier lead removal by the physician. Use of an FEP thermal bond joint is also being studied for Proximal tubing connection on the IS-4 connector. The use of an FEP component thermal bonded to PTFE insulation will likely be used on most future lead designs by Medtronic as a means of achieving strong bond joints in multiple locations that require significant tensile properties. Determine effect of FEP Thermo bond and Polyurethane ring lengths on resulting composite pull forces and suitability of these materials for use as the mPTFE coil liner distal end connection. The goal is to achieve 4.5 lbs. average pull force of the distal end connection.

Through this study it will be determined if the Polyurethane 75D tubing can provide sufficient strength as a rigid member for bonding to the proximal sleevehead while using it in conjunction with thermal bonded FEP segment for NG2 distal design concept.
Two lengths of FEP thermo-bond tubing (0.060/0.090″) were built with two urethane ring lengths (0.060/0.090″) to determine the affect of component length on composite pull strength as potential NG2 distal joint design.
3 Groups of N=30 Samples were assembled using following described method: An FEP tubing segment is thermo-bonded to PTFE coil liner at 800° F. for 16 seconds. A block or tubing is located against proximal side of FEP Tube to hold maintain a square edge on FEP tube during thermo cycle. A silicone tubing over the FEP during thermal bond contains molten FEP and ensures adequate heating of PTFE liner. After thermo processing, silicone tubing is removed and a polyurethane ring is located proximally against FEP segment. An extruded 75D tubing (0.047 I.D/0.005 wall) is bonded onto FEP and urethane ring with tab 006 urethane adhesive to simulate distal sleevehead. Completed subassembly is shown in FIG. 45.
Photo Image of all 4 FEP/Polyurethane Ring sample length combinations are attached as FIG. 46.
Two lengths of FEP thermo-bond (0.060/0.090″) were pull tested with two urethane ring lengths (0.060/0.090″) to understand affect of component length on composite pull strength as potential NG2 distal joint design.
The graphite cylinder tooling used to form edge of FEP during thermal bond resulted in best edge shape as determined by pull test data. The absence of a conductor coil inside the PTFE liner during pull test, may have reduced pull strength by allowing the FEP to pull through urethane ring due to lack of support to PTFE liner while elongating during pull test.
The aluminum block tooling for forming FEP removed excessive heat from PTFE liner during thermo-bond, and caused high occurrence of FEP delamination at a low force pull force. These samples performed worse than other two Sample sets.
The FEP was later pull tested off of PTFE liner at forces of 3.81 to 4.72 lbs between the different component lengths studied, indicating that heat loss during thermal bond was minimal using the silicone tube as tooling method.
Using the polyurethane ring at the 0.060 or 0.090″ length does not have adequate mechanical strength to achieve 4.5 lb. pull force goal due to its inability to prevent FEP from pulling through urethane ring at forces over 3 lbs.

What is claimed is:

1-4. (canceled)

5. A medical electrical lead, comprising:
a lead body;
an inner assembly extending through the lead body including an elongate inner structure forming a lumen enclosing an inner conductor, an elongate conductor extending along an outer surface of the elongate inner structure and a conductive fitting coupled to the clongate conductor at a location thereon that is intermediate the elongate conductor;
an outer insulative layer covering the inner assembly and including an opening in proximity to the fitting, the outer insulative layer having an exterior surface; and an electrode comprising a coil mounted outside the exterior surface of the outer insulative layer and including a feature extending inward through the opening to couple with the conductive fitting;
wherein a buried fitting being incorporated therein.

6. A medical electrical lead, comprising:
a lead body;
an inner assembly extending through the lead body including an elongate inner structure forming a lumen enclosing an inner conductor, an elongate conductor extending along an outer surface of the elongate inner structure and a conductive fitting coupled to the clongate conductor at a location thereon that is intermediate the elongate conductor;
an outer insulative layer covering the inner assembly and including an opening in proximity to the fitting, the outer insulative layer having an exterior surface; and
an electrode comprising a coil mounted outside the exterior surface of the outer insulative layer and including a feature extending inward through the opening to couple with the conductive fitting;
the medical lead being sized less than five French.

7. A medical electrical lead, comprising:
a lead body;
an inner assembly extending through the lead body including an elongate inner structure forming a lumen enclosing an inner conductor, an elongate conductor extending along an outer surface of the elongate inner structure and a conductive fitting coupled to the clongate conductor at a location thereon that is intermediate the elongate conductor;
an outer insulative layer covering the inner assembly and including an opening in proximity to the fitting, the outer insulative layer having an exterior surface; and an electrode comprising a coil mounted outside the exterior surface of the outer insulative layer and including a feature extending inward through the opening to couple with the conductive fitting;
the medical lead being sized less than six French.
